(19) United States
(12) Patent Application Publication
(10) Pub. No.: US 2025/0339389 A1
(43) Pub. Date: Nov. 6, 2025

(54) N-ARACHIDONOYL ETHANOLAMINE COMPOSITION AND USE THEREOF

(71) Applicant: NANJING NUTRABUILDING BIO-TECH CO., LTD., Nanjing (CN)

(72) Inventors: Jing WU, Nanjing (CN); Long JIANG, Nanjing (CN); Shawn WELLS, Nanjing (CN); Kylin LIAO, Nanjing (CN)

(21) Appl. No.: 19/270,336

(22) Filed: Jul. 15, 2025

Related U.S. Application Data
(63) Continuation of application No. PCT/CN2023/072357, filed on Jan. 16, 2023.

(51) Int. Cl. A61K 31/164; A61K 9/00; A61P 1/00

(52) U.S. Cl. CPC A61K 31/164; A61K 9/0053; A61P 1/00

(57) ABSTRACT

The invention discloses an anandamide composition and use thereof in the preparation of nutritional supplements, health-care products, foods and beverages for regulating the intestinal flora. The anandamide composition of the invention can significantly increase level of a first intestinal microbiota in a subject, and simultaneously reduce level of a second intestinal microbiota in the subject, thereby improving the intestinal health.

### CROSS REFERENCE TO RELATED APPLICATION

This application is a continuation application of International Patent Application No. PCT/CN2023/072357, filed on Jan. 16, 2023, the contents of which are incorporated herein by reference in its entirety.

### FIELD OF THE INVENTION

The invention relates to the technical field of health food and dietary supplements, and particularly relates to an anandamide composition and related applications thereof in medicines, nutritional products, dietary supplements, health products, foods and beverages.

### BACKGROUND OF THE INVENTION

There are many long-term colonized microorganisms in human intestine, which can be divided into probiotics, harmful bacteria and conditional pathogenic bacteria according to their relationship with human health. Intestinal flora plays a very important role in human health. It has been proved that flora imbalance is related to the occurrence and development of many metabolic diseases (such as hyperlipidemia and diabetes), cancer and nervous system diseases. Among many influencing factors, diet is the key to shape and adjust the balance of flora. When the diet structure changes, it can quickly detect the changes of diet-related flora diversity, especially the intake of carbohydrates. Therefore, at present, based on different health demands, a variety of microbial regulators (such as antibacterial agents and prebiotics) or live bacteria products have been developed to maintain the body's steady state by changing the composition of the host microbiota (Thursby E, Juge N. Introduction to the human gut microbiota. Biochem J. 2017; 474(11):1823-1836).

Anandamide (N-arachidonoyl ethanolamine, AEA) is a key substance in the human endogenous cannabinoid system, which can interact with intestinal flora to control and regulate various gastrointestinal functions. Endogenous cannabinoid system disorder may cause inflammatory bowel disease, irritable bowel syndrome or obesity. AEA is positively correlated with short-chain fatty acids (such as butyric acid) to achieve anti-inflammatory effect (Vijay A, Kouraki A, Gohir S, et al. The anti-inflammatory effect of bacterial short chain fatty acids is partially mediated by endocannabinoids. Gut Microbes, 2021, 13(1):1997559). Therefore, it is necessary to study the composition and method of supplementing anandamide on intestinal flora in order to maintain the homeostasis of endogenous cannabinoid system and further adjust the balance of intestinal flora to enhance intestinal health.

### SUMMARY OF THE INVENTION

The present invention relates to an anandamide (AEA) composition and use thereof. The invention provides a method for regulating intestinal flora by anandamide, in particular to regulating the levels of probiotics and harmful bacteria in intestinal microbiota and the ratio of probiotics to harmful bacteria. Among them, the probiotics include Roseburia, Bacteroides, Prevotella etc., the harmful bacteria include Salmonella, Escherichia coli etc.

We have found that administration of a composition comprising anandamide or a derivative thereof can regulate intestinal microbiota to enhance intestinal health, including increasing the level of some beneficial microbiota and decreasing the level of some harmful microbiota, and it can relieve pain, promote sleep, stimulate appetite, improve concentration and memory, adjust mood, relieve depression.

To achieve the above objectives, the present invention provides the following technical schemes:

One aspect of the present invention relates to a method for increasing level of a first intestinal microbiota in a subject, wherein the method comprises administering a composition comprising anandamide or a derivative thereof to the subject; the composition comprising anandamide or a derivative thereof effectively increases the level of the first intestinal microbiota in the subject; the first intestinal microbiota is selected from one or more of Roseburia, Bacteroides, Prevotella.

In some embodiments, the method further comprises reducing level of a second intestinal microbiota in the subject, the second intestinal microbiota is Salmonella, Escherichia coli.

In some embodiments, the first intestinal microbiota is a beneficial microbiota.

In some embodiments, the second intestinal microbiota is a harmful microbiota.

In some embodiments, the subject suffers from an intestinal disease.

In some embodiments, the anandamide-containing composition is formulated as nutritional supplement, food, beverage, animal feed or medicine.

In some embodiments, the anandamide-containing composition is prepared into solid preparation or liquid preparation.

In some embodiments, the method comprises administering the composition comprising anandamide or a derivative thereof to the subject at a dosage of 15 to 2000 mg per day.

In some embodiments, the method comprises administering the composition comprising anandamide or a derivative thereof to the subject at a dosage of 30 to 1000 mg per day.

In some embodiments, the anandamide-containing composition is in the form of suppository, tablet, pill, granule, powder, film, capsule, beverage, aerosol, elixir, tincture, tonic, liquid suspension or syrup.

Another aspect of the present invention relates to use of a composition comprising anandamide or a derivative thereof in the preparation of a nutritional supplement, food, beverage, animal feed or medicament for increasing level of a first intestinal microbiota in a subject, wherein the first intestinal microbiota is selected from one or more of Roseburia, Bacteroides, Prevotella.

In some embodiments, the use further comprises reducing level of a second intestinal microbiota in the subject, the second intestinal microbiota is Salmonella, Escherichia coli.

In some embodiments, the first intestinal microbiota is a beneficial microbiota.

In some embodiments, the second intestinal microbiota is a harmful microbiota.

In some embodiments, the use comprises administering the composition comprising anandamide or a derivative thereof to the subject at a dosage of 15 to 2000 mg per day; the composition comprising anandamide or a derivative thereof effectively increases the level of the first intestinal microbiota in the subject.

In some embodiments, the use comprises administering the composition comprising anandamide or a derivative thereof to the subject at a dosage of 1-30 mg/kg body weight per day.

In some embodiments, the subject suffers from an intestinal disease.

In some embodiments, the anandamide-containing composition is prepared into solid preparation or liquid preparation.

In some embodiments, it can relieve pain, promote sleep, stimulate appetite, improve concentration and memory, adjust mood, relieve depression by increasing the level of the first intestinal microbiota in the subject.

In some embodiments, it can relieve pain, promote sleep, stimulate appetite, improve concentration and memory, adjust mood, relieve depression by increasing the level of the first intestinal microbiota in the subject, reducing the level of the second intestinal microbiota in the subject.

In some embodiments, the anandamide-containing composition is in the form of suppository, tablet, pill, granule, powder, film, capsule, beverage, aerosol, elixir, tincture, tonic, liquid suspension or syrup.

The composition comprising anandamide or a derivative thereof can increase the level of Roseburia, which is closely related to intestinal health, and its relative abundance is negatively related to inflammatory bowel disease, irritable bowel syndrome and colorectal cancer. The up-regulation of Roseburia can prevent and treat anxiety and depression-like behaviors, and its level is related to improving concentration and memory and relieving mood. The anandamide-containing composition can also increase the level of Bacteroides, which participate in many important metabolic activities in human colon, including the fermentation of carbohydrates, the utilization of nitrogen-containing substances and the biotransformation of bile acids and other steroids. In addition, it can also produce short-chain fatty acids to maintain steady state and prevent bacterial and viral infections. Up-regulation of Bacteroides can relieve neuropathic pain, and its level is related to relieving emotions. In addition, the anandamide-containing composition can also increase the level of Prevotella. Prevotella is usually regarded as a probiotic related to a healthy plant diet, which helps to decompose protein and carbohydrates, and its abundance is negatively correlated with autism, allergies and multiple sclerosis.

The composition comprising anandamide or a derivative thereof can reduce the level of Escherichia coli, Salmonella. Among them, Escherichia coli is a gram-negative bacterium, which is an important part of the intestinal flora of animals and humans. Most Escherichia coli is non-pathogenic, but it is easy to obtain virulence factors of pathogenic Escherichia coli and become pathogenic bacteria, causing many diseases of animals and people. Salmonella is a common food-borne pathogen. If livestock and poultry are infected with Salmonella, it will cause diseases such as pullorum, swine paratyphoid, avian typhoid and avian paratyphoid, which will reduce the animal's production performance, reproductive capacity and egg laying rate, and even cause acute death. People infected with Salmonella cause acute gastroenteritis, which leads to dizziness, fever, abdominal pain and vomiting, and even death.

### BRIEF DESCRIPTION OF THE FIGURES

FIG. 1 is a graph showing the change of relative abundance ratio of Bacteroides and Firmicutes in feces of three groups of mice.

FIG. 2 is a graph showing changes in the relative abundance of Roseburia in three groups of mice.

FIG. 3 is a graph showing changes in the relative abundance of Bacteroides in three groups of mice.

FIG. 4 is a graph showing changes in the relative abundance of Prevotella in three groups of mice.

FIG. 5 is a graph showing changes in the relative abundance of Salmonella in three groups of mice.

FIG. 6 is a graph showing changes in the relative abundance of Escherichia coli in three groups of mice.

### DETAILED DESCRPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

The present application has found that the administration of anandamide can significantly increase level of a first intestinal microbiota in a subject, and simultaneously reduce level of a second intestinal microbiota in the subject. To this end, the present invention and embodiments thereof are directed to methods using and compositions of “anandamide-containing” compositions and/or “anandamide-enhanced” compositions, wherein anandamide-containing compositions include any composition having anandamide or an anandamide derivative.

As used herein, the terms “subject” or “individual” are used interchangeably to refer to any subject to whom the disclosed methods and compositions can be applied or administered. The subject may have a disease or disorder, but the subject does not need to be sick to benefit from the disclosed methods and compositions. Subjects may need to improve their intestinal and/or overall health, but they may also have overall healthy intestine and wish to maintain or further improve their intestinal and/or overall health. Therefore, any subject can take the disclosed composition or become a recipient of the disclosed method. As used herein, the term “subject” refers to animals (for example, birds, reptiles and mammals). In some embodiments, the subject can be mammals including non-primates (e.g., camels, donkeys, zebras, cows, horses, cats, dogs, rats and mice) and primates (e.g., monkeys, chimpanzees and humans). In certain embodiments, the subject may be a non-human mammal. In other embodiments, the subject may be a human.

The term “administer”, “administering”, or “administration”, as used in this invention, refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

### Composition and Administration

The present invention relates to a method for increasing the level of a first intestinal microbiota and also reducing the level of a second intestinal microbiota in a subject by administering an anandamide-containing composition to the subject at a certain dose.

Depending on the particular anandamide-containing formulation and form, contemplated methods include an administration of at least 15 mg/day of anandamide or an anandamide alternative. This amount per day may be administered at once or in multiple doses. Typically, an effective amount of anandamide or an anandamide derivative in the anandamide-containing composition to be administered at once or in multiple doses per day is of or between 15 milligrams (mg) to 2,000 mg. One or more doses may be administered once a day for any period of time. For example, the effective dose may be administered each day for one day, a few days, multiple days, or on a daily basis indefinitely. More typically, the amount of anandamide or an anandamide derivative to be administered each day is 20 mg, 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg. More typically, the amount of anandamide or an anandamide derivative administered per day is 15 mg to 2000 mg. Most typically, the amount of anandamide or an anandamide derivative administered per day is 200 mg. In certain embodiments, the amount of anandamide administered per day may be about 1-30 mg/kg, about 1-25 mg/kg, about 1-20 mg/kg, about 1-15 mg/kg, about 1-10 mg/kg, about 1-5 mg/kg or about 3-4 mg/kg of body weight.

Multiple forms and formulations of the composition comprising anandamide or a derivative thereof are contemplated for increasing the level of the first intestinal microbiota and also decreasing the level of the second intestinal microbiota in the subject. Compositions will be formulated as a nutritional or dietary supplement, in a (medical) food item, in animal feed, or as a pharmaceutical composition in liquid or solid form comprising the composition comprising anandamide or a derivative thereof, and may optionally also include a nutritionally or pharmaceutically acceptable carrier. For example, where the composition is in solid form, the compositions may be formulated as a snack bar, yogurt, lozenge, tablet, or capsule, or may be coated onto cereal products, included in baked goods. On the other hand, where the supplement is in liquid form, the compositions may be formulated as a tincture, soft gel capsule, liquid capsule, syrup, carbonated drink, a brewed beverage (e.g., as coffee or tea), a juice, an energy drink, a sports drink, or flavored water. While nutritional and pharmaceutical compositions for human use are especially contemplated, it should be appreciated that the composition and formulation comprising anandamide or a derivative thereof may also be employed for veterinary use (e.g., use in animal feed for domestic companion animals (‘pets’) or in animal feed for farm animals.

The composition comprising anandamide or a derivative thereof and its formulation of the invention can be applied to dietary supplements for relieving pain, promoting sleep, stimulating appetite, improving concentration and memory, adjusting mood and relieving depression.

Composition comprising anandamide or a derivative thereof may also be added to a food item comprising microbiota. The anandamide or anandamide derivative may be processed by the consumer concomitantly with the microbiota strain in the food item thereby enhancing the probiotic effects. As used herein, a food item includes any solid or liquid form of food or drink that may be consumed or ingested. As disclosed herein, the amount of the anandamide-containing composition to be added to the food item may vary depending on the kind and form of the food item.

### EXAMPLES

### Example 1

After a week of adaptive feeding, mice were randomly divided into three groups according to sex and weight: group 1, normal group, n=10; group 2, high-fat diet group, n=10; and group 3, high-fat diet+AEA group, n=10. The normal group was given normal feed, while the high-fat diet group was given high-fat and high-sugar feed (containing 35% fat, 18% protein and 47% carbohydrate), and the high-fat diet+AEA group was given high-fat and high-sugar feed containing AEA (20 mg/kg), for four weeks. The specific gavage scheme is: calculate and gavage once a day according to the weight of mice, while the normal group gavage with the same amount of normal saline. Four samples of mouse feces were randomly taken from each group. The samples were first extracted for DNA, 16S rDNA was amplified by PCR, and then sequenced, and the relative abundance of each strain was analyzed.

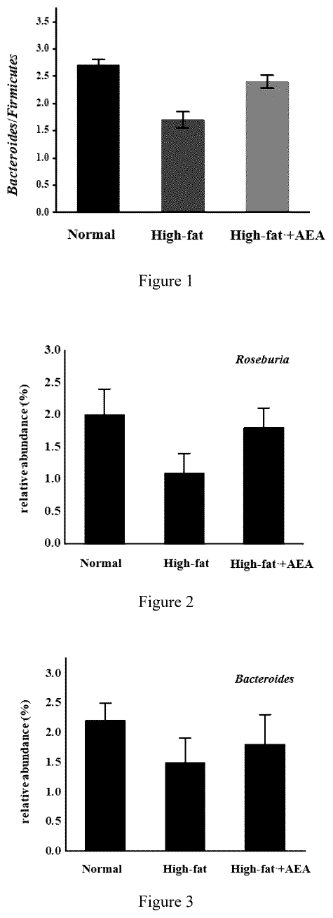

Abundance ratio of Bacteroides and Firmicutes: Bacteroides is the representative of intestinal probiotics and Firmicutes is the representative of intestinal harmful bacteria. The higher the ratio, the healthier the intestinal flora is. The results are shown in FIG. 1. The ratio of Bacteroides/Firmicutes in obese mice induced by high-fat diet in high-fat group decreased significantly, but it increased after AEA administration, which was close to the level of normal group. The above results show that AEA can regulate the structure of intestinal flora and improve the obesity caused by high-fat diet.

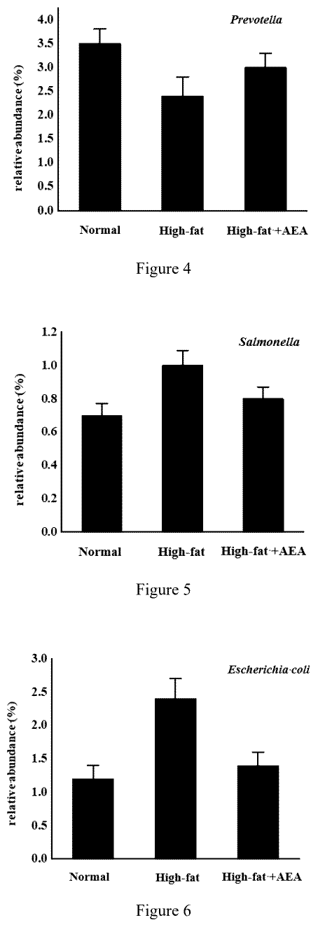

As shown in FIGS. 2-6, compared with normal mice, the flora level of high-fat mice has changed significantly, while the flora level of high-fat mice can be close to that of normal mice by treatment with anandamide (AEA). FIG. 2 is a graph showing changes in the relative abundance of Roseburia. The level of Roseburia in obese mice induced by high-fat diet was significantly lower than that in normal mice, and the level of Roseburia in high-fat+AEA group was close to that in normal mice. FIG. 3 is a graph showing changes in the relative abundance of Bacteroides. The level of Bacteroides in obese mice induced by high-fat diet was significantly lower than that in normal mice. After AEA treatment, the level of Bacteroides in obese mice induced by high-fat diet increased by 20%. FIG. 4 is a graph showing changes in the relative abundance of Prevotella. The level of Prevotella in obese mice induced by high-fat diet was significantly lower than that in normal mice. After AEA treatment, the level of Prevotella in obese mice induced by high-fat diet increased by 25%. FIG. 5 is a graph showing changes in the relative abundance of Salmonella. The Salmonella level of obese mice induced by high-fat diet increased significantly, and after AEA treatment, the Salmonella level of obese mice induced by high-fat diet decreased by 20%. FIG. 6 is a graph showing changes in the relative abundance of Escherichia coli. The level of Escherichia coli in obese mice induced by high-fat diet increased significantly. After AEA treatment, the level of Escherichia coli in obese mice induced by high-fat diet decreased by 42%. The above results show that administration of AEA can increase the level of probiotics in the intestine and reduce the level of harmful bacteria.

### Example 2

Experimental method: Thirty 4-6-week-old mice, 15 males and 15 females, were selected for adaptive feeding for 7 days in advance. They were randomly divided into control group (CON), model group (DSS) and administration group (DSS+AEA), with 10 mice in each group. The model group and the administration group were gavaged with 3% dextran sodium sulfate (DSS) for 9 days to establish an acute colitis model, while the control group was gavaged with the same amount of normal saline every day. The weight, feces and rectal bleeding of mice were recorded every day, and the disease activity index (DAI) was used to evaluate the condition of colitis. The DAI value was obtained by adding all the scores and dividing them by three. The scoring standard is shown in Table 1. Mice in the administration group were given AEA at a daily dose of 2.5 mg/kg. On the 10th day, blood was taken from the orbit of each mouse, and then it was killed. Colon tissues were collected, fixed in 4% paraformaldehyde solution and stored at −80° C. for subsequent analysis. The pathological damage of colon tissue was examined by hematoxylin-eosin staining and Alxin blue staining.

| | [symbol] 1 | DAI [symbol] [symbol] [symbol] | |
|---|---|---|---|
| Weight loss (%) | Feces | Bleeding | Score |
| <1 | normal | none | 0 |
| 1-5 | soft and lumpy | + | 1 |
| 5-10 | wet and soft | ++ | 2 |
| 10-15 | Loose | +++ | 3 |
| >15 | diarrhea-like | massive | 4 |

The results showed that the DAI score of the model group (DSS) mice was close to 3, while the DAI score of the administration group (DSS+AEA) was about 2, which was much lower than that of the model group.

### Example 3

Experimental materials: The experimental animals used in this experiment are male C57BL/6J mice, 6 weeks old, with an initial weight of 18-22 g and SPF grade. The feeding environment of the mice is room temperature of 22±1° C. and humidity of 55±10%, and the mice are fed with normal feed. Mice were randomly divided into blank control group and AEA group, with 10 mice in each group. The AEA group was given a dose of 20 mg/kg/day, while the blank control group was given distilled water of the same volume.

Experimental method: Tail suspension test and forced swimming test were used to evaluate mood and depressive behavior. Forced swimming test: put a single mouse into a beaker and keep the water temperature at (25±2)° C. The experiment lasted for 6 min, and the immobility time of mice within 4 min was calculated. Tail suspension test: a mouse was fixed at the back ⅓ of the tail end with adhesive tape and hung upside down on an iron bar, with its head 10 cm away from the bottom of the box. The experiment lasted for 6 min, and the immobility time of mice within 4 min was calculated.

TABLE 2
Results of forced swimming test and tail suspension test
in mice models of depression in AEA and blank groups

| | Immobility time (min) | |
|---|---|---|
| Group | Forced swimming test | Tail suspension test |
| Blank group | 184.13 ± 29.89 | 121.44 ± 24.91 |
| AEA group | 141.47 ± 25.47 | 87.51 ± 17.69 |

Mice models of forced swimming and tail suspension are widely used in the screening of various antidepressants because of their convenient operation and drug sensitivity. The experimental results are shown in Table 2. The results show that AEA can effectively shorten the immobility time of mice and improve their mood and depression. Among them, in the forced swimming test, the immobility time of AEA group was reduced by 23% compared with the blank group, and in the tail suspension test, the immobility time of AEA group was reduced by 28% compared with the blank group.

### Example 4

Experimental material: The experimental animals used in this experiment are male ICR mice, with an initial weight of 30±2 g and SPF grade. The feeding environment of the mice is room temperature of 22±2° C., and the humidity is controlled at 70%-80%. Mice were free to drink water and eat, and fed with normal feed.

Experimental method: Preparation of animal model: Establishment of SNL model of ligation of the fifth spinal nerve on the left lumbar vertebra in mice: After the animals were anesthetized with isoflurane, the L5 transverse process on the left back was exposed, the L5 nerve was dissociated and ligated with No.6 ophthalmic thread, and the wound was treated with penicillin and then sutured. In Sham group, only L5 transverse process was exposed without nerve ligation. After the model was established, the behavior of mice was observed, and the individuals with impaired motor behavior were excluded. The threshold of 50% mechanical foot contraction of mice left foot was significantly reduced, which indicated that the model was successfully copied. Grouping and administration: Mice were randomly divided into Sham group, model group and AEA group (20 mg/kg/day), with 8 mice in each group. The three groups were administered by gavage for 16 d, wherein the Sham group and model group were given the same volume of distilled water by gavage. Von Frey method was used to detect the mechanical pain sensitivity of mice: after administration, the mice were placed in an organic glass box in a quiet room to adapt for 1.5 h, so that the mice were in a resting state. Von Frey fibers were used to continuously stimulate the L5 nerve reflex area on the sole of the operated hind limb for 3˜4 s, and the test was conducted for 4 times, with an interval of 5 min each time, and the threshold of 50% mechanical foot retraction of the hind paw was recorded.

TABLE 3
Effect of AEA on the threshold of 50% foot contraction in SNL model mice

| | Before | Administration | Administration | Administration | Administration |
|---|---|---|---|---|---|
| Group | modeling | 1 d | 6 d | 11 d | 16 d |
| Sham | 0.389 ± 0.035 | 0.345 ± 0.025 | 0.360 ± 0.034 | 0.373 ± 0.014 | 0.364 ± 0.028 |
| group | | | | | |
| SNL | 0.369 ± 0.037 | 0.005 ± 0.002 | 0.019 ± 0.008 | 0.018 ± 0.018 | 0.020 ± 0.018 |
| model | | | | | |
| AEA | 0.323 ± 0.040 | 0.010 ± 0.010 | 0.067 ± 0.013 | 0.133 ± 0.023 | 0.193 ± 0.002 |
| group | | | | | |

Compared with the Sham group, the threshold of 50% foot retraction in SNL model group was significantly reduced; compared with the SNL model, the threshold of 50% foot contraction in AEA group increased significantly after 6 days of administration.

In the invention, after the administration of anandamide, the content of other beneficial bacteria can be increased, such as Bifidobacterium, Lactobacillus rhamnosus, Clostridium butyricum, etc. Meanwhile, the content of other harmful bacteria has decreased, such as Staphylococcus aureus, Clostridium perfringens, Enterococcus, etc., which makes the level of these intestinal microbiota close to the normal level and is very beneficial to maintaining intestinal health.

The foregoing is only the preferred examples of the present invention, and is not intended to limit the present invention. Any person skilled in this field can make many changes, modifications, substitutions and variations on these examples without departing from the principles and purposes of the present invention. The scope of the invention is defined by the claims and their equivalents.

What is claimed is:

1. A method for increasing level of a first intestinal microbiota in a subject, wherein the method comprises administering a composition comprising anandamide or a derivative thereof to the subject; the composition comprising anandamide or a derivative thereof effectively increases the level of the first intestinal microbiota in the subject; the first intestinal microbiota is selected from one or more of Roseburia, Bacteroides, Prevotella.

2. The method of claim 1, wherein the method further comprises reducing level of a second intestinal microbiota in the subject, the second intestinal microbiota is Salmonella, Escherichia coli.

3. The method of claim 1, wherein the first intestinal microbiota is a beneficial microbiota.

4. The method of claim 2, wherein the second intestinal microbiota is a harmful microbiota.

5. The method of claim 1, wherein the anandamide-containing composition is formulated as nutritional supplement, food, beverage, animal feed or medicine.

6. The method of claim 1, wherein the anandamide-containing composition is prepared into solid preparation or liquid preparation.

7. The method of claim 1, wherein the composition comprising anandamide or a derivative thereof is administered to the subject at a dosage of 15 to 2000 mg per day.

8. The method of claim 1, wherein the composition comprising anandamide or a derivative thereof is administered to the subject at a dosage of 1-30 mg/kg body weight per day; the anandamide-containing composition effectively increases the level of the first intestinal microbiota in the subject.

9. The method of claim 1, wherein it can relieve pain, promote sleep, stimulate appetite, improve concentration and memory, adjust mood, relieve depression by increasing the level of the first intestinal microbiota in the subject.

10. The method of claim 1, wherein it can relieve pain, promote sleep, stimulate appetite, improve concentration and memory, adjust mood, relieve depression by increasing the level of the first intestinal microbiota in the subject, and reducing level of a second intestinal microbiota in the subject.

11. The method of claim 10, wherein the first intestinal microbiota is selected from one or more of Roseburia, Bacteroides, Prevotella, the second intestinal microbiota is Salmonella, or Escherichia coli.

12. The method of claim 1, wherein the subject suffers from an intestinal disease.

13. The method of claim 1, wherein the anandamide-containing composition is in the form of suppository, tablet, pill, granule, powder, film, capsule, beverage, aerosol, elixir, tincture, tonic, liquid suspension or syrup.
